(19) United States
(12) Patent Application Publication
(10) Pub. No.: US 2024/0382353 A1
(43) Pub. Date: Nov. 21, 2024

(54) FEMININE HYGIENE PAD WITH FOAM ABSORBENT LAYER COMPONENT AND IMPROVED BODY CONFORMITY

(71) Applicant: The Procter & Gamble Company, Cincinnati, OH (US)

(72) Inventors: Holly Balasubramanian RAUCKHORST, Fort Thomas, KY (US); Digvijay RAWAT, Cincinnati, OH (US)

(21) Appl. No.: 18/666,976

(22) Filed: May 17, 2024

Related U.S. Application Data
(60) Provisional application No. 63/503,173, filed on May 19, 2023.

(51) Int. Cl. A61F 13/538

(52) U.S. Cl. CPC A61F 13/538; A61F 2013/5383

(57) ABSTRACT

A feminine hygiene pad comprises a liquid-permeable topsheet, a liquid-impermeable backsheet, and an absorbent layer disposed between the topsheet and the backsheet. The absorbent layer comprises a layer of open-celled foam comprising an arrangement of macro-voids therein. The arrangement of macro-voids defines paths that lie along an x-y planar surface of the layer of open-celled foam and comprise left and right forward path legs disposed predominantly forward of the lateral axis.

### CROSS REFERENCE TO RELATED APPLICATIONS

This application claims the benefit, under 35 U.S.C. § 119 (e), to U.S. Provisional Application No. 63/503,173, filed May 19, 2023, the entire disclosure of which is fully incorporated by reference herein.

### FIELD OF THE INVENTION

The present disclosure generally relates to a feminine hygiene pad with a foam absorbent layer component, in particular, a feminine hygiene pad with a foam absorbent layer having an arrangement of macro-voids.

### BACKGROUND OF THE INVENTION

Feminine hygiene pads including absorbent components formed of resilient, open-celled polymeric foam are currently manufactured and marketed. The inclusion of a resilient open-celled foam is deemed desirable to some consumer/user market segments because it can impart a cushiony, compliant and comfortable feel to the pad, and in some examples, provide for a pad that is relatively thin (i.e., has a relatively low caliper), which is desirable because a thin and compliant pad can be relatively discreet when worn under outer clothing, as compared with pads that rely upon cellulosic fiber material components for absorbency.

For feminine hygiene pads, close conformity of the pad to the wearer's body is deemed important for at least three purposes: minimization of chances that discharged menstrual fluid can migrate along skin surfaces and escape being captured and absorbed by the pad (and thereby soil underwear, outer clothing, bedclothes, etc.); maximization of efficient utilization of the absorbent components and functions of the pad; and discreetness under outer clothing. Due to the resilience of typical open-celled polymeric foams, however, pads manufactured with such foam layer components may be somewhat resistive to flexing and creasing, and may have shape memory biasing them toward their as-manufactured configuration (typically, generally planar/flat). As a result, they may be somewhat resistive to maintaining close conformity with the complex, non-ruled curving and intersecting shape features of the human female body in the crotch region. Absorbent products having more structured construction, e.g., with 3D shape features that provide for closer body conformity (such as interlabial pads or inserts) may be a cause or source of discomfort.

Consequently, a manufacturer that can provide a comfortable, compliant feminine hygiene pad having improved body conformity, compared to products currently available, may enjoy a competitive advantage in the marketplace.

### SUMMARY OF THE INVENTION

The present disclosure solves the problem of feminine hygiene pad body conformity by providing an absorbent layer with an arrangement of macro-voids which can improve the ability of the pad to conform closely to a wearer's body in the crotch region, while also remaining comfortable for the wearer.

Described herein is a feminine hygiene pad comprising a forward end, a rearward end, and a longitudinal axis and a lateral axis, the axes having an intersection; a liquid-permeable topsheet; a liquid-impermeable backsheet; and an absorbent layer disposed between the topsheet and the backsheet. The absorbent layer comprises a layer of open-celled foam having a z-direction caliper and an outer perimeter along an x-y plane. The layer of open-celled foam comprises an arrangement of macro-voids therein, the macro-voids having a z-direction depth. The arrangement of macro-voids defines paths that lie along an x-y planar surface of the layer of open-celled foam and comprise a left forward path leg disposed predominantly forward of the lateral axis and beginning at a left central location proximate the intersection of the axes, and ending at a left outboard location disposed forward of the first left central location, and further outboard of the longitudinal axis than the first left central location; and a right forward path leg disposed predominantly forward of the lateral axis and beginning at a right central location proximate the intersection of the axes, and ending at a right outboard location disposed forward of the first right central location, and further outboard of the longitudinal axis than the first right central location.

Also described herein is a feminine hygiene pad comprising a forward end, a rearward end, and a longitudinal axis and a lateral axis, the axes having an intersection; a liquid-permeable topsheet; a liquid-impermeable backsheet; and an absorbent layer disposed between the topsheet and the backsheet. The absorbent layer comprises a layer of open-celled foam having a z-direction caliper and an outer perimeter along an x-y plane. The layer of open-celled foam comprises an arrangement of macro-voids therein, the macro-voids having a z-direction depth. The arrangement of macro-voids defines paths that lie along an x-y planar surface of the layer of open-celled foam and comprise a left forward path leg disposed predominantly forward of the lateral axis and beginning at a left central location proximate the intersection of the axes, and ending at a left outboard location disposed forward of the first left central location, and further outboard of the longitudinal axis than the first left central location; and a right forward path leg disposed predominantly forward of the lateral axis and beginning at a right central location proximate the intersection of the axes, and ending at a right outboard location disposed forward of the first right central location, and further outboard of the longitudinal axis than the first right central location. At least one of the left forward path leg and the right forward path leg has a ratio of a macro-void length to a gap length of from about 30:1 to about 1:5.

### BRIEF DESCRIPTION OF THE DRAWINGS

FIG. 1 is a plan view of an example of a feminine hygiene pad, topsheet side facing the viewer.

FIG. 2 is a plan view of an example of an absorbent layer.

FIG. 3A is a schematic lateral cross section of the feminine hygiene pad of FIG. 1.

FIG. 3B is an enlarged portion 3B of the drawing of FIG. 3A, enlarged to depict sublayers of an absorbent layer, which may be present in some examples.

FIG. 4A is a plan view of an example of an absorbent layer, with an example of an arrangement of macro-voids.

FIGS. 4B and 4C are plan views of the absorbent layer shown in FIG. 4A, illustrating paths of the macro-voids.

FIG. 4D is a plan view of the absorbent layer shown in FIG. 4A, illustrating zones of z-direction bending or displacement about paths of macro-voids.

FIGS. 4E and 4F are lateral cross sections taken along lines 4E-4E and 4F-4F shown in FIG. 4D, in configurations the absorbent layer would assume when the pad including it is worn by a wearer/user.

FIG. 4G is a plan view of an example of an absorbent layer, with an example of an arrangement of macro-voids combined with examples of arrangements of perforations.

FIG. 4H is a plan view of another example of an absorbent layer, with an example of an arrangement of macro-voids combined with examples of arrangements of perforations.

FIG. 4I is a plan view of an example of an absorbent layer, with an example of an arrangement of macro-voids and an added stiffener.

FIG. 5 is an illustration of an example of an arrangement of macro-voids defining a forward path as described herein.

FIG. 6 is an illustration of an arrangement of macro-voids not defining a forward path as described herein.

FIG. 7 is an illustration of an arrangement of macro-voids not defining a forward path as described herein.

FIG. 8 is an illustration of an example of an arrangement of macro-voids defining a forward path as described herein.

FIG. 9 is an illustration of an arrangement of macro-voids not defining a forward path as described herein.

FIG. 10 is an illustration of an example of an arrangement of macro-voids defining a rearward path as described herein.

FIGS. 11A and 11B are schematic cross-section illustrations of a portion of a pad with an absorbent layer uncreased (FIG. 11A) and creased (FIG. 11B) across a full-depth macro-void.

FIG. 12 is a schematic cross-section illustration of a portion of a pad with an absorbent layer having a partial-depth macro-void.

### DETAILED DESCRIPTION

### Definitions

With respect to a feminine hygiene pad that is opened and laid out flat on a horizontal planar surface, “lateral” refers to a direction perpendicular to the longitudinal direction and parallel to the horizontal planar surface.

With respect to a feminine hygiene pad that is opened and laid out flat on a horizontal planar surface and having a length measured from forward end to rearward end, “longitudinal” refers to a direction parallel with the line along which the length is measured, and parallel to the horizontal planar surface. “Length” refers to a dimension measured in the longitudinal direction.

With respect to a feminine hygiene pad, the terms “front,” “rear,” “forward” and “rearward” relate to features or regions of the pad corresponding to the position it would occupy as ordinarily worn by a user, and the front/anterior and rear/posterior of the user's body when standing.

With respect to a feminine hygiene pad that is opened and laid out flat on a horizontal planar surface, or a nonwoven web material laid out flat on a horizontal planar surface, “z-direction” refers to a direction perpendicular to the horizontal planar surface, and any plane parallel to the horizontal planar surface may be referred to as an “x-y plane”. When the pad is being worn by a user (and thus has been urged into a curving configuration), “z-direction” at any particular point location on the pad refers to a direction generally normal to the wearer-facing surface of the pad at the particular point location. With respect to a nonwoven web during its manufacture, “z-direction” refers to a direction orthogonal to both the machine direction and the cross direction of manufacture, and any plane parallel to the machine direction and cross direction may be referred to as an “x-y plane”.

With respect to a feminine hygiene pad, “wearer-facing” is a relative locational term referring to a feature of a component or structure of the pad that when in use that lies closer to the wearer than another feature of the component or structure that lies along the same z-direction. For example, a topsheet has a wearer-facing surface that lies closer to the wearer than the opposite, outward-facing surface of the topsheet.

With respect to a feminine hygiene pad, “outward-facing” is a relative locational term referring to a feature of a component or structure of the pad that when in use lies farther from the wearer than another feature of the component or structure that lies along the same z-direction. For example, a topsheet has an outward-facing surface that lies farther from the wearer than the opposite, wearer-facing surface of the topsheet.

The relative location terms “inboard” and “outboard” refer to positioning of a first feature relative to a second feature with respect to the lateral or longitudinal axis of a pad or layer component thereof, when both features are on the same side of the axis. For example, a first feature is laterally “inboard” of a second feature, and the second feature is laterally “outboard” of the first feature, when the first feature is closer to the longitudinal axis of the pad than the second feature. Similarly, a first feature is longitudinally “inboard” of a second feature, and the second feature is longitudinally “outboard” of the first feature, when the first feature is closer to the lateral axis of the pad than the second feature.

A “macro-void” in a foam layer is a defined void in the layer, having x-y planar and z-direction dimensions, that is visible at arms' length distance to the naked eye of an adult person with normal vision, that has been created by either (a) removal of material from the foam, via manufacturing process, following formation of the foam; or (b) by molding of the foam precursor material(s) to mold the macro-void into the finished foam. Accordingly, a closed or open cell of random size and location occurring within a cellular foam structure as a feature of a foam-making process is not a “macro-void” for purposes herein. A macro-void may extend entirely through the z-direction caliper of the foam layer, or it may extend only partially through the z-direction caliper of the foam layer.

A “forward path of macro-voids” (or similar expression) refers to any identifiable imaginary straight line or curved path along the x-y plane of a surface of an absorbent layer, having each of a starting point and an ending point at a distal edge of a macro-void, wherein the starting and ending points are at least 2.5 cm apart, and along which at least 50 percent, preferably at least 67 percent, and more preferably at least 75 percent of the length there along traverses two or more macro-voids, and includes no discontinuity or change of direction greater than 30 degrees, from a starting point to an ending point 2.5 cm or less apart along a straight line dimension.

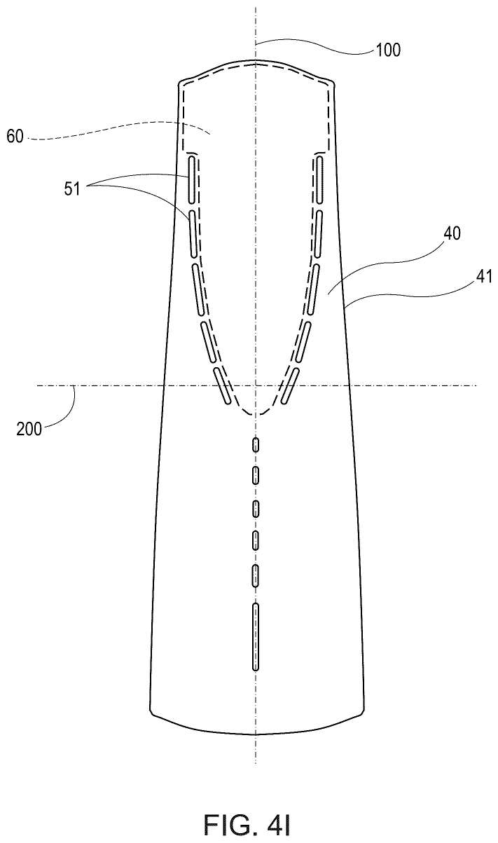

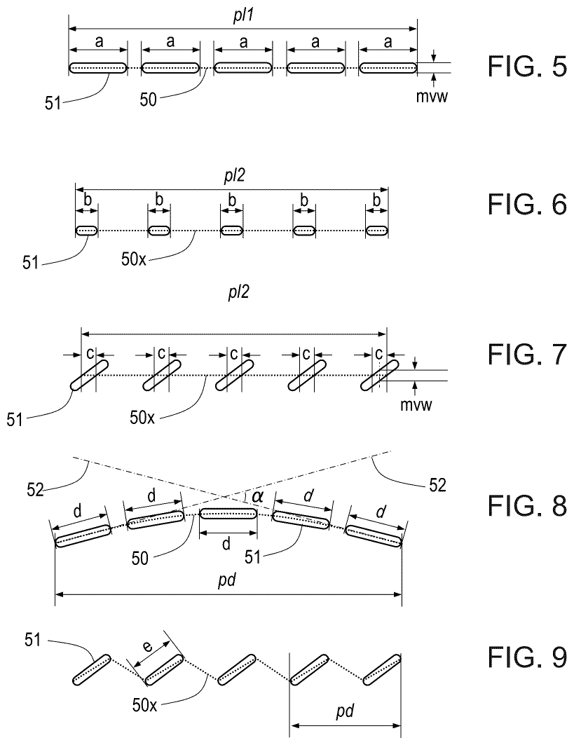

To illustrate, FIG. 5 depicts a straight-line forward path 50f (shown as a dotted line) of macro-voids 51 conforming to this definition. Path 50f has a starting point on the distal edge of the left-most macro-void in the figure, and an ending point on the distal edge of the right-most macro-void in the figure. The length of the path is pl1; and more than 40 percent of that length traverses the five macro-voids 51 shown, such that the (sum of the lengths a)/pl1>0.50.

FIG. 6 depicts a path 50x that does not conform to this definition, because the (sum of the lengths b)/pl2<0.50.

FIG. 7 depicts a path 50x that does not conform to this definition, because the (sum of the lengths c)/pl3<0.50.

In FIG. 8, assuming that straight-line path dimension pd is greater than 2.5 cm and smaller angle α between lines 52 tangent to the curve of the forward path 50f at each of its ends is no greater than 30 degrees, FIG. 8 depicts a curved forward path 50f of macro-voids 51 conforming to this definition. The path does not change in direction by more than 30 degrees along a straight-line path dimension pd greater than 2.5 cm in length. The sum of the individual lengths d of the macro-voids 51 traversed by the path, divided by the length of the curved forward path 50f, is greater than 0.50.

In FIG. 9, an alternate attempt to identify a conforming path of macro-voids similar to those shown in FIG. 7 is illustrated. As the proposed path 50x is drawn, the portion of it that traverses macro-voids 51 each having a largest dimension e, is maximized and the portion that does not traverse macro-voids 51 is minimized. The sum of the largest dimensions e divided by the total path length is now greater than 0.50. However, assuming that straight-line path dimension pd shown is less than 2.5 cm, FIG. 9 depicts a path 50x that does not conform to this definition, because the path changes in direction by more than 30 degrees (and does so twice) within dimension pd that is less than 2.5 cm. This disqualifying change in direction is repeated through the entirety of proposed path 50x. Thus, even as re-drawn, proposed path 50x does not conform to this definition.

A “rearward path of macro-voids” (or similar expression) refers to any identifiable imaginary straight line or curved path along the x-y plane of a surface of an absorbent layer, having each of a starting point and an ending point at a distal edge of a macro-void, wherein the starting and ending points are at least 2.5 cm apart, and along which at least 30 percent, or at least 40 percent, or at least 60 percent of the length there along traverses two or more macro-voids, and includes no discontinuity or change of direction greater than 30 degrees, from a starting point to an ending point 2.5 cm or less apart along a straight line dimension.

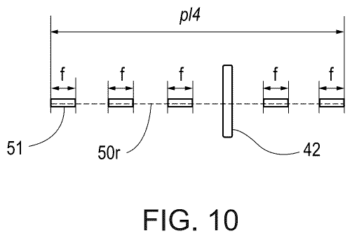

FIG. 10 depicts a path 50r that conforms to this definition, because the (sum of the lengths f)/pl4>0.30. As shown in FIG. 10, in some configurations, a portion of the rearward path of macro-voids may intersect one or more perforations 42r (as described herein).

### DESCRIPTION

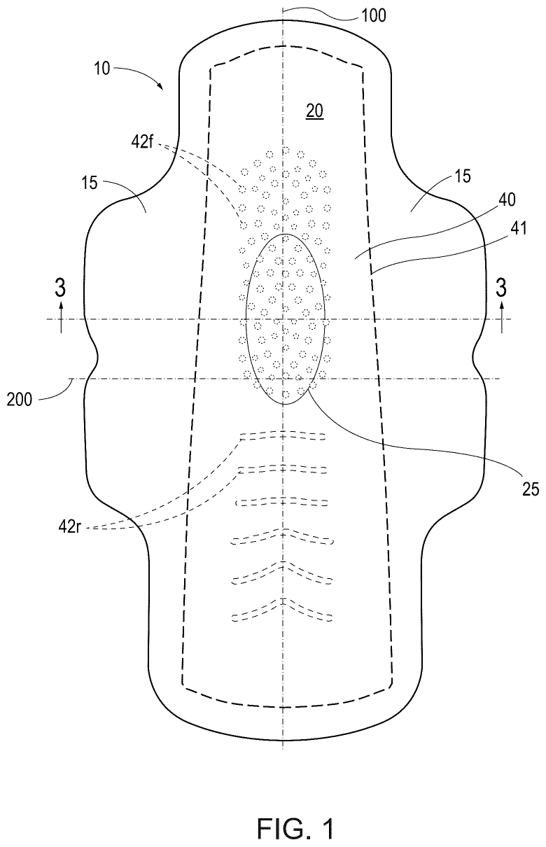

Referring to FIGS. 1, 2, and 3A, a feminine hygiene pad 10 may include a liquid permeable topsheet 20, a liquid impermeable backsheet 30 and an absorbent layer 40 disposed between the topsheet and the backsheet. The absorbent layer has an outer perimeter 41. In regions outside the outer perimeter 41, the topsheet and the backsheet may be bonded together in laminated fashion by any suitable mechanism including but not limited to adhesive bonding, thermal bonding, pressure bonding, etc., thereby enveloping, retaining and holding the absorbent layer 40 in place between the topsheet 20 and the backsheet 30. Absorbent layer 40 may be cut or otherwise imparted with a shape that is asymmetric about the lateral axis, as suggested in the figures, for purposes of enabling the cutting away of consecutive absorbent layers 40 from stock material along nested profiles, providing for efficient absorbent layer stock material utilization/minimization of cutoff scrap. It may be preferred that the laterally wider portion of the shape is in the rear of the pad, for purposes of providing more surface area to intercept fluid that moves along skin through the gluteal crevice. Pad 10 may include opposing wing portions 15 extending laterally outside of perimeter 41 by a comparatively greater width dimension than the main portion of the pad. The outer surface of the backsheet forming the undersides of the main portion and the wing portions may have deposits of fastening adhesive 35 thereon. Fastening adhesive deposits 35 may be provided to enable the user to adhere the pad to the inside of her underpants in the crotch region thereof, and wrap the wing portions through and around the inside edges of the leg openings of the underpants and adhere them to the outside/underside of the underpants in the crotch region, providing supplemental holding support and helping guard the leg edges of the underpants against soiling. When pad 10 is packaged, fastening adhesive deposits 35 may be covered by one or more sheets of release film or paper (not shown) that covers/shields the fastening adhesive deposits 35 from contact with other surfaces and/or contamination, until the user is ready to remove the release film or paper and place the pad for use.

### Topsheet

Topsheet 20 may be formed of any suitable nonwoven web material that is compliant, soft feeling, and non-irritating to wearers' skin. Referring back to the figures, the topsheet 20 is positioned adjacent a wearer-facing surface of the absorbent layer 40 and may be joined thereto and to the backsheet 30 by any suitable attachment or bonding method. The topsheet 20 and the backsheet 30 may be joined directly to each other in the peripheral regions outside the perimeter 41 of the absorbent layer 40 and may be indirectly joined by directly joining them respectively to wearer-facing and outward-facing surfaces of the absorbent layer or additional optional layers included with the pad.

A suitable topsheet material will include a liquid pervious material that is comfortable when in contact with the wearer's skin and permits discharged menstrual fluid to rapidly penetrate through it. A suitable topsheet may be made of various materials such as nonwoven web materials.

Non-limiting examples of nonwoven web materials that may be suitable for use as the topsheet include fibrous materials made from natural fibers, modified natural fibers, synthetic fibers, or combinations thereof. Some suitable examples are described in U.S. Pat. Nos. 4,950,264; 4,988,344; 4,988,345; 3,978,185; 7,785,690; 7,838,099; 5,792,404; and 5,665,452.

In some examples, the topsheet may comprise tufts as described in U.S. Pat. Nos. 8,728,049; 7,553,532; 7,172,801; 8,440,286; 7,648,752; and 7,410,683. The topsheet may have a pattern of discrete hair-like fibrils as described in U.S. Pat. No. 7,655,176 or U.S. Pat. No. 7,402,723. Additional examples of suitable topsheet materials include those described in U.S. Pat. Nos. 8,614,365; 8,704,036; 6,025,535 and US 2015/041640. Another suitable topsheet may be formed from a three-dimensional substrate as detailed in US 2017/0258647. The topsheet may have one or more layers, as described in US 2016/0167334; US 2016/0166443; and US 2017/0258651.

As contemplated herein, component nonwoven web material from which topsheet 20 may be cut may be a nonwoven web material that includes or consists predominately (by weight) of fibers spun from polymeric resin such as polyolefins and/or polyesters, including but not limited to polypropylene, polyethylene and variants, blends, and bicomponent or multicomponent arrangements thereof.

The nonwoven web may be formed via any suitable process by which spun fibers of indefinite lengths may be distributed and accumulated in a controlled fashion onto a moving forming belt to form a batt having a desired distribution of the fibers, to a desired basis weight. Suitable processes may include spunbonding and meltblowing. After accumulation, the batt may be processed to consolidate and bond the fibers into a cohesive web by any suitable method, including calendering, calender thermal bonding, calender compression bonding, through-air bonding, etc. The consolidated web may be subjected to further processes such as hydroenhancing or hydroentangling, to increase z-direction entanglement of fibers, and increase loft.

In some configurations, the nonwoven web material may be formed in a co-forming process in which hydrophilic fibers (such as plant-based, e.g., cotton fibers, rayon fibers, etc.) of finite lengths are physically blended or mixed with streams of spun fibers of longer but indefinite lengths, spun from polymeric resin, and laid down on a forming belt to form a web as described in, for example, U.S. Pat. Nos. 8,017,534; 4,100,324; US 2003/0200991; U.S. Pat. No. 5,508,102; US 2003/0211802; EP 0 333 228; WO 2009/10938; US 2017/0000695; US 2017/0002486; U.S. Pat. No. 9,944,047; 2017/0022643 and US 2018/0002848.

Absent enhancements to the materials and/or processes involved, generally, monocomponent fibers spun from polymer resin tend to have relatively simple surface geometry, typically a round or approximately oval-shaped cross section, and a substantially non-curled or non-crimped configuration along the lengths thereof. As a consequence, when the spun fibers are deposited and accumulated on a forming belt, calendered and bonded (e.g., in a spunbonding process), the resulting nonwoven web product will have relatively low loft and a relatively flat appearance, as compared with a web of a comparable basis weight formed of more complexly-shaped, e.g., curled or crimped, fibers. A lower loft nonwoven web may be perceived by some consumers to have a comparatively less pleasing feel and appearance, i.e., it may be perceived to be, relatively, not as soft or luxurious, as a higher-loft one.

To add loft to the web without increasing basis weight (and material usage), and to increase opacity of the web, the fibers used to make the web may be spun in multicomponent, e.g., bicomponent fiber configurations. Resin-processing equipment and beams of spinnerets may be configured, and polymer resins may be selected, to spin bicomponent fibers that crimp or curl as they leave the spinnerets as molten polymer streams, and subsequently cool and solidify into fibers. Known processes and polymer resin selections may be used to produce curly spun bicomponent fibers wherein the fibers have side-by-side, eccentric core-sheath, or other non-coaxial polymer component cross-sectional configurations. In such non-coaxial configurations, one of the polymer components may be selected and/or formulated to have a differing melting temperature and/or cooling contraction rate than the other polymer component. Upon cooling, the differing properties of the polymer components and non-coaxial cross-sectional arrangement of component sections of the molten fiber streams impart curl to the fibers as they cool, contract at differing rates and solidify. The respective polymer resin components may be differing polymers, differing forms or variants of the same polymers, or differing blends thereof. More detailed disclosure of spinning curled or crimped bicomponent fibers and forming a nonwoven web thereof may be found in, for example, U.S. Pat. No. 8,501,646; US EP 1 988 793; and US 2007/0275622. In some examples bicomponent fibers may have respective predominately polypropylene-based resin components formulated to impart differing melting temperatures to the respective components. In some examples bicomponent fibers may have respective components in which one component is predominately polypropylene-based, and the other component is predominately polyethylene-based. In some more particular examples the bicomponent fibers may be spun with an eccentric core-sheath component configuration wherein a predominately polypropylene-based component is the core component and a predominately polyethylene-based component is the sheath component; wherein the polypropylene-based component may be desired for its greater tensile strength, and the polyethylene-cased component may be desired for its smoother, more lubricious surface feel, that helps impart a silky feel to the fiber and to the nonwoven web material. It will be appreciated that other combinations of polyolefins and/or other spinnable thermoplastic resins may be selected for their differing cooling contraction rates and other differing qualities that affect the qualities (including curl or crimp) and properties of the spun fibers in differing ways.

The topsheet may further incorporate or include any features of topsheets described in U.S. patent application Ser. Nos. 16/789,516 and/or 16/789,522.

Many commercially practical thermoplastic resins that may be desired to process and spin into bicomponent fibers are normally hydrophobic. Such resins include polyolefins such as polypropylene and polyethylene. A nonwoven web material formed of such fibers will also be hydrophobic, and as such, will not readily accept or wick aqueous fluid such as menstrual fluid. When such resins are used, therefore, additional measures can be included to render the fibers and/or the nonwoven web hydrophilic. In some configurations, a suitable surfactant may be applied to the nonwoven web following its formation. A particularly suitable surfactant finish used may be SILASTOL PHP 26, a product of Schill+Seilacher GmbH, Böblingen, Germany. The finish may be applied to the web using any suitable method, for example, via kiss roll coater. The finish may be applied in a quantity suitable to impart the nonwoven web with a desired level of hydrophilicity and thereby help impart it with a desired level of capillary absorption/desorption pressure. In some configurations, a finish coating of SILASTOL PHP 26 may be applied in a quantity sufficient to constitute, after drying, surfactant weight quantity that is 0.30 percent to 0.60 percent, more preferably 0.40 percent to 0.50 percent, of the basis weight of the nonwoven web material.

Wicking performance also may vary according to, and may be manipulated by, the manner in which the web is further processed. Factors such as level of consolidation (i.e., densification) of the fiber mass in the end structure and orientations of the individual fibers within the end structure can affect absorbency and wicking performance.

Thus, for purposes contemplated herein, in combination with being imparted with a suitable basis weight, density and/or caliper as discussed above, it may be desired that nonwoven web material formed in part or in substantial entirety of fibers spun from thermoplastic polymer resin and used to make the topsheet, be formed via a nonwoven web manufacturing process in which substantial portions of the fibers are imparted with directional orientation that includes some z-direction orientation, rather than orientations predominately biased along the machine direction or x-y plane of formation of the web structure. Following any suitable processes in which fibers are distributed and laid down in a batt on a horizontal forming belt (e.g., via a spunbond process), additional process steps that forcibly reorient some of the fibers or portions thereof in the z-direction may be employed. Suitable process steps may include needle punching and hydroentangling or hydroenhancing. Hydroentangling or hydroenhancing, in which an array of fine, high-velocity water jets is directed at the batt as it is conveyed past them, may be desired for its effectiveness in reorienting lengths of fibers while breaking fewer fibers and creating less broken fiber lint and surface fuzz (free fiber ends extending from the surface of the web). A vacuum water removal system (in which air is drawn through the web in a z-direction into a pattern of orifices or pores on a vacuum drum or belt conveying the batt, pulling the jetted water with it) may be desired because it tends to create, add, open and/or clear small z-direction passageways within the fiber matrix of the web, approximately in the pattern of the orifices or pores. Without intending to be bound by theory, it is believed that the portions of the fibers oriented in the z-direction and the z-direction passageways increase the ability and tendency of the web to wick aqueous fluid in the z-direction. In a topsheet, this would mean that the material can more readily wick aqueous fluid from the wearer-facing surface of the topsheet to the outward-facing surface of the topsheet, i.e., down to the absorbent layer below, and may thereby wick fluid less along x-y planar directions (causing a stain from discharged fluid to spread laterally and/or longitudinally).

### Absorbent Layer

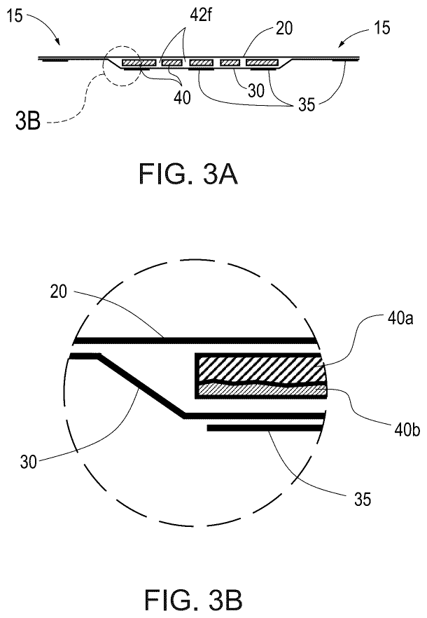

In some configurations, the absorbent layer 40 may be formed of or include a layer of absorbent open-celled foam material. The foam material may include at least first and second sublayers 40a, 40b (FIG. 3B) of absorbent open-celled foam material, the sublayers being in direct face-to-face contact with each other. In such configurations, the wearer-facing sublayer may be a relatively larger-celled foam material, and the outward-facing sublayer may be a relatively smaller-celled foam material, for purposes explained in more detail below.

The open-celled foam material may be a foam material that is manufactured via polymerization of the continuous oil phase of a water-in-oil high internal phase emulsion (“HIPE”).

A water-in-oil HIPE has two phases. One phase is a continuous oil phase comprising monomers to be polymerized, and an emulsifier to help stabilize the HIPE. The oil phase may also include one or more photoinitiators. The monomer component may be included in an amount of from about 80% to about 99%, and in certain examples from about 85% to about 95%, by weight of the oil phase. The emulsifier component, which is soluble in the oil phase and suitable for forming a stable water-in-oil emulsion may be included in the oil phase in an amount of from about 1% to about 20%, by weight of the oil phase. The emulsion may be formed at an emulsification temperature of from about 20° C. to about 130° C.

In general, the monomers may be included in an amount of about 20% to about 97% by weight of the oil phase and may include at least one substantially water-insoluble monofunctional alkyl acrylate or alkyl methacrylate. For example, monomers of this type may include C4-C18 alkyl acrylates and C2-C18 methacrylates, such as ethylhexyl acrylate, butyl acrylate, hexyl acrylate, octyl acrylate, nonyl acrylate, decyl acrylate, isodecyl acrylate, tetradecyl acrylate, benzyl acrylate, nonyl phenyl acrylate, hexyl methacrylate, 2-ethylhexyl methacrylate, octyl methacrylate, nonyl methacrylate, decyl methacrylate, isodecyl methacrylate, dodecyl methacrylate, tetradecyl methacrylate, and octadecyl methacrylate.

The oil phase may also include from about 2% to about 40, by weight of the oil phase, a substantially water-insoluble, polyfunctional crosslinking alkyl acrylate or methacrylate. This crosslinking comonomer, or crosslinker, is added to confer strength and resilience to the resulting HIPE foam. Examples of crosslinking monomers of this type comprise monomers containing two or more activated acrylate, methacrylate groups, or combinations thereof. Nonlimiting examples of this group include 1,6-hexanedioldiacrylate, 1,4-butanedioldimethacrylate, trimethylolpropane triacrylate, trimethylolpropane trimethacrylate, 1,1 2-dodecyldimethacrylate, 1,14-tetradecanedioldimethacrylate, ethylene glycol dimethacrylate, neopentyl glycol diacrylate (2,2-dimethylpropanediol diacrylate), hexanediol acrylate methacrylate, glucose pentaacrylate, sorbitan pentaacrylate, and the like.

Any third substantially water-insoluble comonomer may be added to the oil phase in weight percentages of from about 0% to about 15% by weight of the oil phase, to modify properties of the HIPE foams. In certain cases, “toughening” monomers may be desired to impart toughness to the resulting HIPE foam. These include monomers such as styrene, vinyl chloride, vinylidene chloride, isoprene, and chloroprene. Without being bound by theory, it is believed that such monomers aid in stabilizing the HIPE during polymerization (also known as “curing”) to provide a more homogeneous and better-formed HIPE foam which results in greater toughness, tensile strength, abrasion resistance, and the like. Monomers may also be added to confer flame retardancy, as disclosed, for example, in U.S. Pat. No. 6,160,028. Monomers may be added to impart color (for example vinyl ferrocene); to impart fluorescent properties; to impart radiation resistance; to impart opacity to radiation (for example lead tetraacrylate); to disperse charge; to reflect incident infrared light; to absorb radio waves; to make surfaces of the HIPE foam struts or cell walls wettable; or for any other desired property in a HIPE foam. In some cases, these additional monomers may slow the overall process of conversion of HIPE to HIPE foam, the tradeoff being necessary if the desired property is to be conferred. Thus, such monomers can also be used to slow down the polymerization rate of a HIPE. Examples of monomers of this type comprise styrene and vinyl chloride.

The oil phase may further include an emulsifier to stabilize the HIPE. Emulsifiers used in a HIPE can include: (a) sorbitan monoesters of branched C16-C24 fatty acids; linear unsaturated C16-C22 fatty acids; and linear saturated C12-C14 fatty acids, such as sorbitan monooleate, sorbitan monomyristate, and sorbitan monoesters, sorbitan monolaurate diglycerol monooleate, polyglycerol monoisostearate, and polyglycerol monomyristate; (b) polyglycerol monoesters of—branched C16-C24 fatty acids, linear unsaturated C16-C22 fatty acids, or linear saturated C12-C14 fatty acids, such as diglycerol monooleate (for example diglycerol monoesters of C18:1 fatty acids), diglycerol monomyristate, diglycerol monoisostearate, and diglycerol monoesters; (c) diglycerol monoaliphatic ethers of—branched C16-C24 alcohols, linear unsaturated C16-C22 alcohols, and linear saturated C12-C14 alcohols, and mixtures of these emulsifiers. See U.S. Pat. Nos. 5,287,207 and 5,500,451. Another emulsifier that may be used is polyglycerol succinate, which is formed from an alkyl succinate, glycerol, and triglycerol.

Such emulsifiers, and combinations thereof, may be added to the oil phase so that they constitute about 1% to about 20% of the weight of the oil phase. In some configurations, coemulsifiers may also be used to provide additional control of cell size, cell size distribution, and emulsion stability, particularly at higher temperatures, for example greater than about 65° C. Examples of coemulsifiers include phosphatidyl cholines and phosphatidyl choline-containing compositions, aliphatic betaines, long chain C12-C22 dialiphatic quaternary ammonium salts, short chain C1-C4 dialiphatic quaternary ammonium salts, long chain C12-C22 dialkoyl(alkenoyl)-2-hydroxyethyl, short chain C1-C4 dialiphatic quaternary ammonium salts, long chain C12-C22 dialiphatic imidazolinium quaternary ammonium salts, short chain C1-C4 dialiphatic imidazolinium quaternary ammonium salts, long chain C12-C22 monoaliphatic benzyl quaternary ammonium salts, long chain C12-C22 dialkoyl(alkenoyl)-2-aminoethyl, short chain C1-C4 monoaliphatic benzyl quaternary ammonium salts, short chain C1-C4 monohydroxyaliphatic quaternary ammonium salts. In some configurations, ditallow dimethyl ammonium methyl sulfate may be used as a coemulsifier.

Any photoinitiators included may be included at between about 0.05% and about 10%, by weight of the oil phase. Lower amounts of photoinitiator may allow light to better penetrate the HIPE foam, which can provide for polymerization deeper into the HIPE foam. However, if polymerization is performed in an oxygen-containing environment, it may be desired that there be enough photoinitiator present to initiate the polymerization and overcome oxygen inhibition. Photoinitiators can respond rapidly and efficiently to a light source with the production of radicals, cations, and other species that are capable of initiating a polymerization reaction. Photoinitiators selected for use in forming foams within contemplation of the present disclosure may absorb UV light at wavelengths of about 200 nanometers (nm) to about 800 nm, in certain examples about 250 nm to about 450 nm. If the photoinitiator is in the oil phase, suitable types of oil-soluble photoinitiators include benzyl ketals, α-hydroxyalkyl phenones, α-amino alkyl phenones, and acylphospine oxides. Non-limiting examples of suitable photoinitiators can include 2,4,6-[trimethylbenzoyldiphosphine]oxide in combination with 2-hydroxy-2-methyl-1-phenylpropan-1-one (50:50 blend of the two is sold by Ciba Specialty Chemicals, Ludwigshafen, Germany as DAROCUR 4265); benzyl dimethyl ketal (sold by Ciba Geigy as IRGACURE 651); α-,α-dimethoxy-α-hydroxy acetophenone (sold by Ciba Speciality Chemicals as DAROCUR 1173); 2-methyl-1-[4-(methyl thio)phenyl]-2-morpholino-propan-1-one (sold by Ciba Speciality Chemicals as IRGACURE 907); 1-hydroxycyclohexyl-phenyl ketone (sold by Ciba Speciality Chemicals as IRGACURE 184); bis(2,4,6-trimethylbenzoyl)-phenylphosphineoxide (sold by Ciba Speciality Chemicals as IRGACURE 819); diethoxyacetophenone, and 4-(2-hydroxyethoxy)phenyl-(2-hydroxy-2-methylpropyl) ketone (sold by Ciba Speciality Chemicals as IRGACURE 2959); and Oligo [2-hydroxy-2-methyl-1-[4-(1-methylvinyl)phenyl]propanone] (sold by Lamberti spa, Gallarate, Italy as ESACURE KIP EM).

The dispersed aqueous phase of a HIPE comprises water, and may also comprise one or more components, such as initiator, photoinitiator, or electrolyte, wherein in certain examples, the one or more components are at least partially water soluble.

One component included in the aqueous phase may be a water-soluble electrolyte. The water phase may contain from about 0.2% to about 40%, by weight of the aqueous phase of a water-soluble electrolyte. The electrolyte minimizes the tendency of monomers, comonomers, and crosslinkers that are primarily oil soluble to also dissolve in the aqueous phase. Examples of electrolytes include chlorides or sulfates of alkaline earth metals such as calcium or magnesium and chlorides or sulfates of alkali metals such as sodium. Such electrolyte can include a buffering agent for the control of pH during the polymerization, including such inorganic counterions as phosphate, borate, and carbonate, and mixtures thereof.

Another component that may be included in the aqueous phase is a water-soluble free-radical initiator. The initiator can be present at up to about 20 mole percent based on the total moles of polymerizable monomers present in the oil phase. In certain examples, the initiator may be included in an amount of from about 0.001 to about 10 mole percent based on the total moles of polymerizable monomers in the oil phase. Suitable initiators may include ammonium persulfate, sodium persulfate, potassium persulfate, 2,2′-azobis(N,N′-dimethyleneisobutyramidine)dihydrochloride, azo initiators, redox couples like persulfate-bisulfate, persulfate-ascorbic acid, and other suitable redox initiators. In some configurations, to reduce the potential for premature polymerization which may clog the emulsification system, addition of the initiator to the monomer phase may be performed near the end of the emulsification step, or shortly afterward.

Photoinitiator included in the aqueous phase may be at least partially water soluble, and may constitute between about 0.05% and about 10%, by weight of the aqueous phase. Lower amounts of photoinitiator may allow light to better penetrate the HIPE foam, which can provide for polymerization deeper into the HIPE foam. However, if polymerization is done in an oxygen-containing environment, there should be enough photoinitiator to initiate the polymerization and overcome oxygen inhibition. Photoinitiators can respond rapidly and efficiently to a light source with the production of radicals, cations, and other species that are capable of initiating a polymerization reaction. Photoinitiators selected for use to form foams within contemplation of the present disclosure may absorb UV light at wavelengths of from about 200 nanometers (nm) to about 800 nm, in certain examples from about 200 nm to about 350 nm, and in certain examples from about 350 nm to about 450 nm. If a photoinitiator is to be included in the aqueous phase, suitable types of water-soluble photoinitiators may include benzophenones, benzils, and thioxanthones. Examples of photoinitiators include 2,2′-Azobis [2-(2-imidazolin-2-yl)propane]dihydrochloride; 2,2′-Azobis [2-(2-imidazolin-2-yl)propane]disulfate dehydrate; 2,2′-Azobis(1-imino-1-pyrrolidino-2-ethylpropane)dihydrochloride; 2,2′-Azobis [2-methyl-N-(2-hydroxyethyl) propionamide]; 2,2′-Azobis(2-methylpropionamidine)dihydrochloride; 2,2′-dicarboxymethoxydibenzalacetone, 4,4′-dicarboxymethoxydibenzalacetone, 4,4′-dicarboxymethoxydibenzalcyclohexanone, carboxymethoxydibenzalacetone; and 4,4′-disulphoxymethoxydibenzalacetone. Other suitable 4-dimethylamino-4′-photoinitiators that can be used are listed in U.S. Pat. No. 4,824,765.

In addition to the previously described components, other components may be included in either the aqueous or oil phase of a HIPE. Examples include antioxidants, for example hindered phenolics, hindered amine light stabilizers; plasticizers, for example dioctyl phthalate, dinonyl sebacate; flame retardants, for example halogenated hydrocarbons, phosphates, borates, inorganic salts such as antimony trioxide or ammonium phosphate or magnesium hydroxide; dyes and pigments; fluorescers; filler particles, for example starch, titanium dioxide, carbon black, or calcium carbonate; fibers; chain transfer agents; odor absorbers, for example activated carbon particulates; dissolved polymers; dissolved oligomers; and the like.

HIPE foam is produced from the polymerization of the monomers comprising the continuous oil phase of a HIPE. In certain examples, a HIPE foam layer may have one or more sublayers, and may be either homogeneous or heterogeneous polymeric open-celled foams. Homogeneity and heterogeneity relate to distinct layers within the same HIPE foam, which are similar in the case of homogeneous HIPE foams and differ in the case of heterogeneous HIPE foams. A heterogeneous HIPE foam may contain at least two distinct sublayers that differ with regard to their chemical composition, physical properties, or both; for example, sublayers may differ with regard to one or more of foam density, polymer composition, specific surface area, or pore size (also referred to as cell size). For example, for a HIPE foam if the difference relates to pore size, the average pore size in the respective sublayers may differ by at least about 20%, in certain examples by at least about 35%, and in still other examples by at least about 50%. In another example, if the differences in the sublayers of a HIPE foam layer relate to density, the densities of the layers may differ by at least about 20%, in certain examples by at least about 35%, and in still other examples by at least about 50%. For instance, if one layer of a HIPE foam has a density of 0.020 g/cc, another layer may have a density of at least about 0.024 g/cc or less than about 0.016 g/cc, in certain examples at least about 0.027 g/cc or less than about 0.013 g/cc, and in still other examples at least about 0.030 g/cc or less than about 0.010 g/cc. If the differences between the layers are related to the chemical composition of the HIPE or HIPE foam, the differences may reflect a relative amount difference in at least one monomer component, for example by at least about 20%, in certain examples by at least about 35%, and in still further examples by at least about 50%. For instance, if one sublayer of a HIPE or HIPE foam is composed of about 10% styrene in its formulation, another sublayer of the HIPE or HIPE foam may be composed of at least about 12%, and in certain examples of at least about 15%.

A HIPE foam layer structured to have distinct sublayers formed from differing HIPEs may provide a HIPE foam layer with a range of desired performance characteristics. For example, a HIPE foam layer comprising first and second foam sublayers, wherein the first foam sublayer has a relatively larger pore or cell size, than the second sublayer, when used in an absorbent article may more quickly absorb incoming fluids than the second sublayer. For example, when the HIPE foam layer is used to form an absorbent layer of a feminine hygiene pad, the first foam sublayer may be layered over the second foam sublayer having relatively smaller pore sizes, as compared to the first foam sublayer, which exert more capillary pressure and draw the acquired fluid from the first foam sublayer, restoring the first foam sublayer's ability to acquire more fluid from above. HIPE foam pore sizes may range from 1 to 200 μm and in certain examples may be less than 100 μm. HIPE foam layers of the present disclosure having two major parallel surfaces may be from about 0.5 to about 10 mm thick, and in certain examples from about 2 to about 10 mm. The desired thickness of a HIPE foam layer will depend on the materials used to form the HIPE foam layer, the speed at which a HIPE is deposited on a belt, and the intended use of the resulting HIPE foam layer.

The HIPE foam layers of the present disclosure are relatively open-celled. This refers to the individual cells or pores of the HIPE foam layer being in substantially unobstructed communication with adjoining cells. The cells in such substantially open-celled HIPE foam structures have intercellular openings or windows that are large enough to permit ready fluid transfer from one cell to another within the HIPE foam structure. For purpose of the present disclosure, a HIPE foam is considered “open-celled” if at least about 80% of the cells in the HIPE foam that are at least 1 μm in size are in fluid communication with at least one adjoining cell.

In addition to being open-celled, in certain examples HIPE foams are adapted to be sufficiently hydrophilic to permit the HIPE foam to absorb aqueous fluids. In some examples the internal surfaces of a HIPE foam may be rendered hydrophilic by residual hydrophilizing surfactants or salts left in the HIPE foam following polymerization, by selected post-polymerization HIPE foam treatment procedures (as described hereafter), or combinations of both.

In some configurations, for example when it is used to form an absorbent layer of a feminine hygiene pad, a HIPE foam layer may be flexible and exhibit an appropriate glass transition temperature (Tg). The Tg represents the midpoint of the transition between the glassy and rubbery states of the polymer. In general, HIPE foams that have a Tg that is higher than the temperature of use can be strong but will also be relatively rigid and potentially prone to fracture (brittle). In certain examples, regions of the HIPE foams of the current disclosure which exhibit either a relatively high Tg or excessive brittleness will be discontinuous. Since these discontinuous regions will also generally exhibit high strength, they can be prepared at lower densities without compromising the overall strength of the HIPE foam.

HIPE foams intended for applications requiring flexibility should contain at least one continuous region having a Tg as low as possible, so long as the overall HIPE foam has acceptable strength at in-use temperatures. In certain examples, the Tg of this region will be less than about 40° C. for foams used at about ambient temperature conditions; in certain other examples Tg will be less than about 30° C. For HIPE foams used in applications wherein the use temperature is higher or lower than ambient temperature, the Tg of the continuous region may be no more than 10° C. greater than the use temperature, in certain examples the same as use temperature, and in further examples about 10° C. less than use temperature wherein flexibility is desired. Accordingly, monomers are selected as much as possible that provide corresponding polymers having lower Tg's.

HIPE foams useful for forming absorbent layers and/or sublayers within contemplation of the present disclosure, and methods for their manufacture, also include but are not necessarily limited to those foams and methods described in U.S. Pat. Nos. 10,045,890; 9,056,412; 8,629,192; 8,257,787; 7,393,878; 6,551,295; 6,525,106; 6,550,960; 6,406,648; 6,376,565; 6,372,953; 6,369,121; 6,365,642; 6,207,724; 6,204,298; 6,158,144; 6,107,538; 6,107,356; 6,083,211; 6,013,589; 5,899,893; 5,873,869; 5,863,958; 5,849,805; 5,827,909; 5,827,253; 5,817,704; 5,817,081; 5,795,921; 5,741,581; 5,652,194; 5,650,222; 5,632,737; 5,563,179; 5,550,167; 5,500,451; 5,387,207; 5,352,711; 5,397,316; 5,331,015; 5,292,777; 5,268,224; 5,260,345; 5,250,576; 5,149,720; 5,147,345; and US 2005/0197414; US 2005/0197415; US 2011/0160326; US 2011/0159135; US 2011/0159206; US 2011/0160321; and US 2011/0160689, which are incorporated herein by reference to the extent not inconsistent herewith.

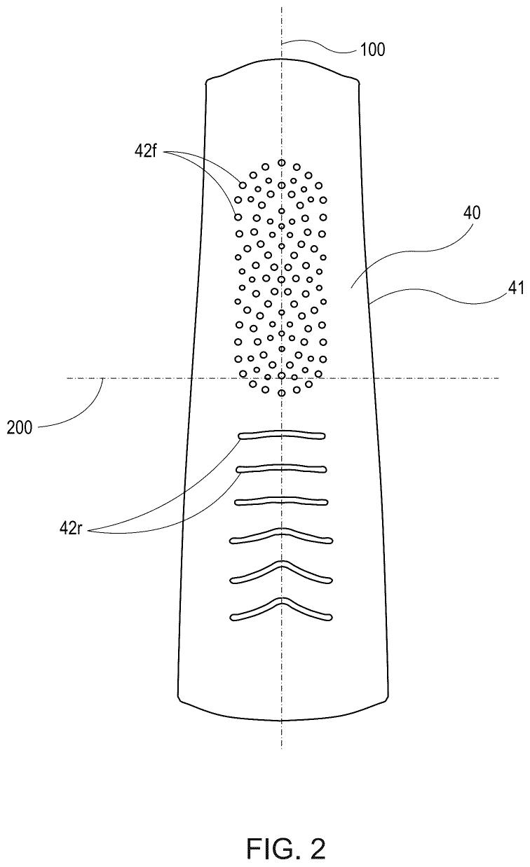

As reflected in FIGS. 1 and 2, the absorbent layer formed of HIPE foam may include one or more patterns of forward and rearward perforations 42f, 42r, including at least a first pattern disposed within an expected discharge location proximate the intersection of longitudinal and lateral axes 100, 200 of the pad. Perforations 42f, 42r may be punched, cut, molded, or otherwise formed through the entire z-direction depth of the HIPE foam absorbent layer, or only through a wearer-facing layer or partially into the wearer-facing portion thereof. When a HIPE foam absorbent layer is disposed in direct contact with a topsheet as described herein, with no intervening acquisition layer formed of another material, perforations 42f, 42r may serve as a group of “reservoirs” to receive, temporarily hold, and aid in distributing rapid discharges of relatively small quantities of menstrual fluid, until the HIPE foam has sufficient time to distribute and absorb the fluid via capillary action. Additionally, such perforations help decrease bending stiffness of the absorbent layer, which may help increase comfort of the pad for the wearer. A pattern of perforations having an average radius or other largest dimension of 1.0 mm to 4.0 mm, and more preferably 1.5 mm to 3.5 mm may be included, within, for example, the area occupied by a bonding region 25. The pattern may include perforations at a numerical density of 3.0 to 9.0 perforations per cm², and more preferably 4.0 to 8.0 perforations per cm². In selecting the appropriate average size, numerical density, and surface area occupied by the pattern of perforations, the manufacturer may wish to balance the volume of the “reservoirs” desired with the need to retain absorbent material in locations proximate to and about the expected discharge location. Additional details concerning configurations of such perforations in combination with examples of suitable absorbent layers may be found in U.S. Pat. No. 8,211,078.

Preferably, the topsheet 20 is bonded to the wearer-facing surface of the absorbent layer 40 in a manner that assures close proximity between the two, to provide for rapid fluid movement down through the topsheet to the absorbent layer 40, while not creating an unacceptable degree of occlusion (created, e.g., by overly large deposits of adhesive between these components) that would obstruct downward fluid movement. Bonding the topsheet to the absorbent layer also helps fix the absorbent layer 40 within the enveloped space, and helps unitize the overall structure of the pad, enhancing user/wearer impressions of quality. The topsheet 20 may be bonded to the absorbent layer 40 in any suitable manner, including that described in U.S. patent application Ser. No. 16/789,522. By effectively unitizing the absorbent layer 40 and topsheet 20, such bonding also may help reduce any potential for the pad to fold or crease along series of perforations, and thereby further direct and promote hinging or folding as contemplated herein, along paths of macro-apertures. To further unitize the pad and minimize development of open space (space not occupied by absorbent material) between the topsheet and backsheet, the outward-facing surface of the absorbent layer may also be bonded to the backsheet via a deposited pattern or dispersion of adhesive applied between these components, to either the wearer-facing surface of the backsheet 30 or the outward-facing surface of absorbent layer 40.

### Macro-Voids

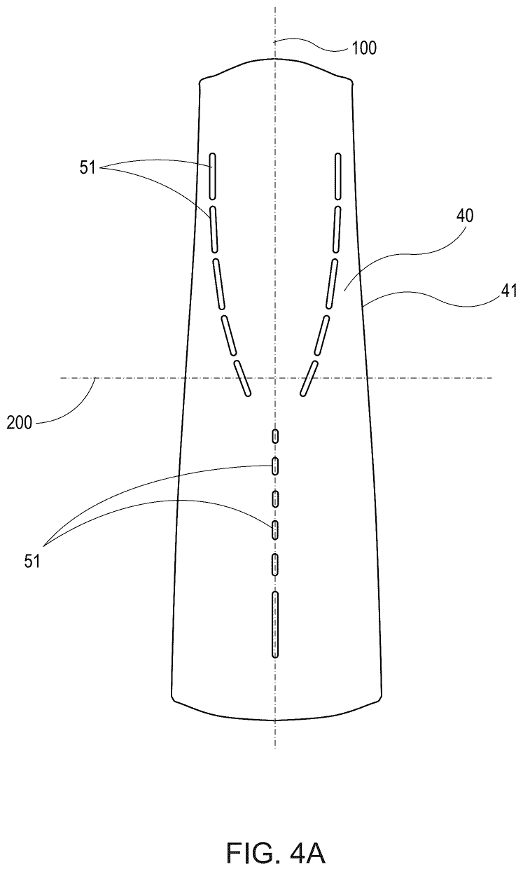

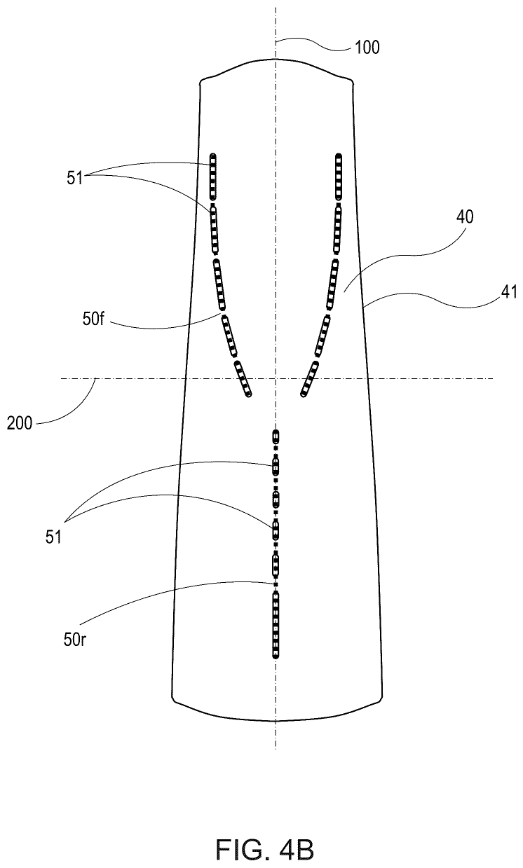

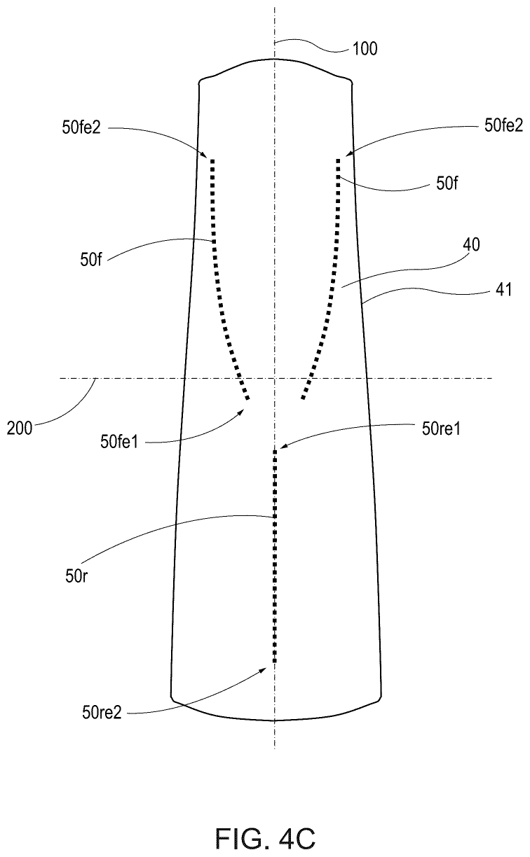

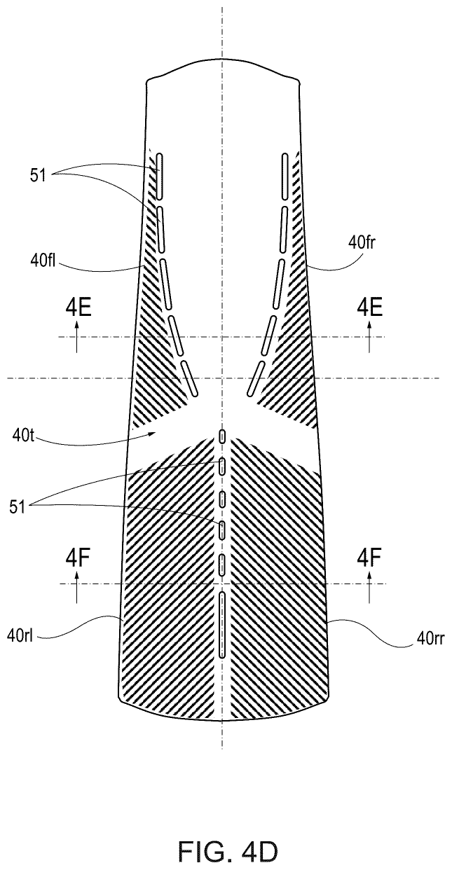

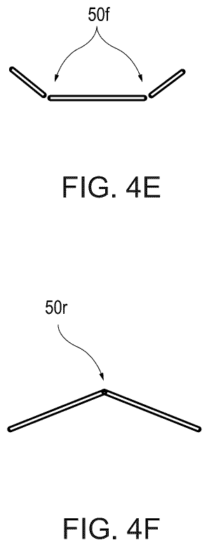

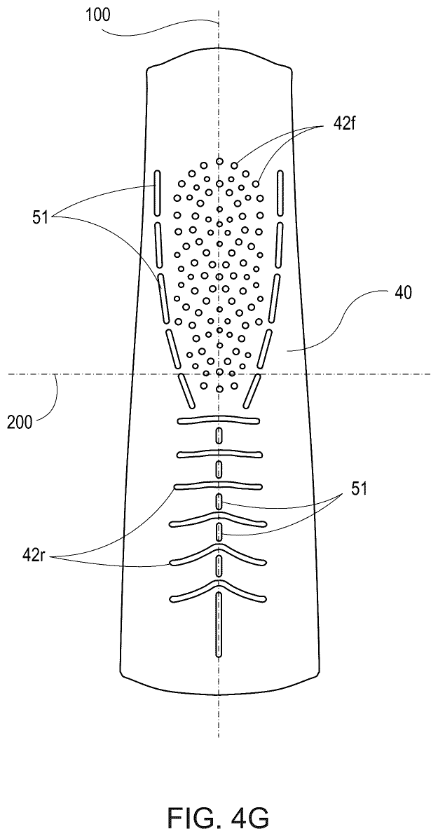

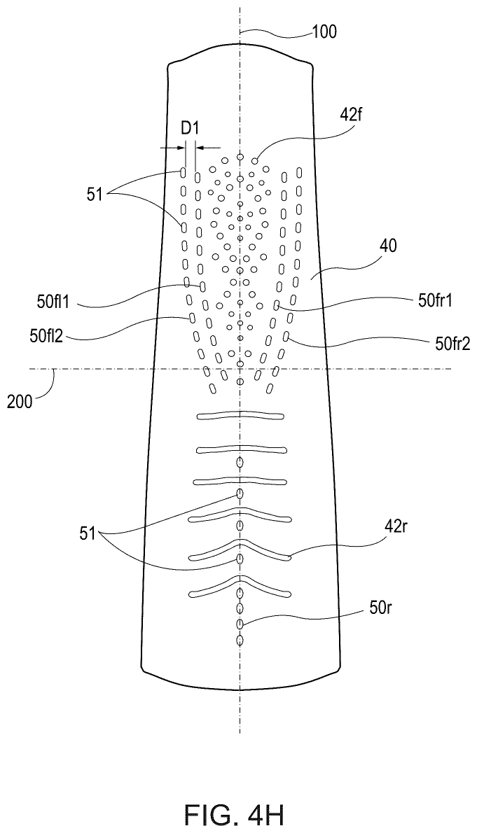

Referring to FIGS. 4A-4D, an absorbent layer 40 may be provided with an arrangement of macro-voids 51. As contemplated herein, macro-voids 51 may have similarities to perforations 42f and 42r as described above, in that they are visible voids formed in the absorbent layer 40. They are differentiated for purposes contemplated herein, however, in that macro-voids 51 serve to facilitate or promote generally longitudinal creasing or folding of the absorbent layer 40. In contrast, forward perforations 42f serve as fluid reservoirs as described above, and rearward perforations 42r serve as fluid reservoirs or channels, and also facilitate or promote generally lateral bending/curvature of the pad upwardly about the wearer's buttocks in the rear. In some configurations, for macro-voids 51 to more reliably function as described below, it may be desired that macro-voids 51 have an x-y planar aspect ratio of at least 1.5:1, more preferably at least 2:1, with the longer dimension thereof substantially oriented along a path such as path 50f, 50r.

It has been learned that imparting an absorbent layer 40 with an arrangement of macro-voids, having features described herein, can dramatically improve the ability of the pad 10 to conform closely to a wearer's body in the crotch region, while also remaining comfortable for the wearer.

The suitable arrangement may include paths of macro-voids 51 that include left and right forward paths 50f substantially symmetrically arranged about and on either side of longitudinal axis 100, and a rearward path 50r substantially centered along and/or substantially following longitudinal axis 100. A left forward path leg may be disposed predominantly forward of the lateral axis 200 and begin at a left central location proximate the intersection of the lateral axis 200 and the longitudinal axis 100, and end at a left outboard location disposed forward of the first left central location, and further outboard of the longitudinal axis 100 than the first left central location. A right forward path leg may be disposed predominantly forward of the lateral axis 200 and begin at a right central location proximate the intersection of the lateral axis 200 and the longitudinal axis 100, and end at a right outboard location disposed forward of the first right central location, and further outboard of the longitudinal axis 100 than the first right central location. A rearward path leg may be disposed predominantly rearward of the lateral axis 200.

Each of left and right forward paths 50f may have a first endpoint 50fe1 located relatively closer to the longitudinal axis 100 and closer to the intersection of axes 100, 200 (herein, the “center”) of the pad, than a second endpoint 50fe2. The rearward path 50r may have a first endpoint 50re1 located relatively closer to the lateral axis 200, than a second endpoint 50re2. It may be desired that the left and right forward paths 50f do not completely converge or intersect, e.g., at a location on or proximate the longitudinal axis 100, so as to retain structural integrity of the foam layer by avoiding increasing the likelihood of tear propagation beyond the paths, which might result from manipulation or movement of the foam layer, during pad manufacture, pad handling, or pad use/wear. It may be desired that the forward paths 50f do not meet or intersect the rearward path 50r, for similar reasons.

Referring specifically to FIGS. 4C-4F, through prototyping and testing it has been learned that an arrangement of macro-voids 51 having these features facilitates creasing or hinging of the absorbent layer 40 about the paths 50f, 50r. This may be more readily achievable when the absorbent layer 40 is formed of a pliable foam, such as a polyurethane foam or HIPE foam as described herein. Through testing it has been learned that, when a pad having some or any combination of these features is suitably located and applied to a user/wearer's underpants and then normally worn, zones 40fl and 40fr tend to flex upwardly (or toward the wearer's body) about creases formed along paths 50f of macro-voids 51, causing the pad to assume a cupped configuration (in lateral cross section) that better conforms to the wearer's body in the region proximate the vagina. (See FIG. 4E.) At the same time, zones 40rl and 40rr tend to flex downwardly (or away from the wearer's body) about a crease formed along path 50r of macro-voids 51, causing the pad to assume an inverted-V-shaped configuration (in lateral cross section) that better conforms to the wearer's body along the gluteal groove. (See FIG. 4F.) When the absorbent layer 40 is formed of a suitable foam such as polyurethane or HIPE foam, the pliability and elasticity of the foam may better allow the absorbent layer 40 to inflect and transition between these two configurations in transition zone 40t.

Referring to FIGS. 4G and 4H, these conformability tendencies may be synergistically enhanced by inclusion of and combination with patterns of perforations 42f and 42r, as described above. A suitable pattern of apertures 42f such as, for example, the pattern shown in FIGS. 4G and 4H, located predominantly or entirely between left and right forward paths 50f, can serve not only the reservoir and distribution functions described above, but can also increase flexibility and pliability of the absorbent layer 40 in that zone, enhancing its ability to cup about the user/wearer's body in the region proximate the vagina.

A suitable pattern of apertures 42r, can cooperate with macro-voids 51 in the rearward portion of the absorbent layer 40, to enable the rearward portion of layer 40 to curve and wrap upwardly about the wearer's buttocks as viewed from a side, and, at the same time, crease along longitudinal axis 100 to better conform to the wearer's body within and proximate the gluteal groove. It will be appreciated that attempting to urge a generally flat/planar material to conform to such complexly curved and intersecting surfaces may otherwise be difficult.

In some configurations, it may be preferred that paths of macro-voids 51 traverse or encompass more than two, more preferably more than three, even more preferably more than four, and even more preferably more than five macro-voids 51, with areas of absorbent layer material continuity between the macro-voids. In some configurations, it may be preferred that a path of macro-voids 51 traverses or encompasses from about 2 to about 18 macro-voids, with areas of absorbent layer material continuity between the macro-voids 51, or from about 4 to about 15 macro-voids 51, or from about 6 to about 12 macro-voids 51, or from about 8 to about 10 macro-voids 51. Providing areas of material continuity between successive macro-voids 51 along a path reduces the ability or prevents the macro-void from channeling fluid there along (which may have undesirable consequences), and may also help the absorbent layer retain structural robustness and avoid unwanted tearing thereof, while still facilitating desired creasing along the path. In some configurations, the area of material continuity between successive macro-voids 51 along a path may have a gap length (G) of from about 2 mm to about 6 mm, or from about 3 mm to about 5 mm.

Where generally laterally-oriented perforations 42r (as described herein) are included with a path 50r of macro-voids 51 as described herein, it may be desired that the macro-voids 51 and laterally-oriented perforations 42r do not intersect or coincide, i.e., that areas of material continuity are present between macro-voids 51 and perforations 42r, again, for purposes of retaining structural robustness of the absorbent layer 40.

As shown in FIG. 4H, in some configurations, it may be preferred to have more than one left and/or right forward paths 50f of macro-voids. For instance, a suitable arrangement may comprise a first left forward path 50fl1 and a second left forward path 50f12 and/or a first right forward path 50fr1 and a second right forward path 50fr2. In some configurations, the distance (D1) between the first left forward path 50fl1 and the second left forward path 50f12 and/or between the first right forward path 50fr1 and the second right forward path 50fr2 can be from about 2 mm to about 6 mm, or from about 3 mm to about 4 mm.

In some configurations, the macro-voids of the first left forward path 50fl1 and the second left forward path 50f12 may be staggered. In some configurations, the macro-voids of the first right forward path 50fr1 and the second right forward path 50fr2 may be staggered. The first left forward path 50fl1 and the second left forward path 50f12 and/or the first right forward path 50fr1 and the second right forward path 50fr2 may be substantially the same length or may be different lengths. In some configurations, the second left forward path 50f12 may comprise more macro-voids and/or may be longer than the first left forward path 50fl1 or vice versa. In some configurations, the second right forward path 50fr2 may comprise more macro-voids and/or may be longer than the first right forward path 50fr1 or vice versa.

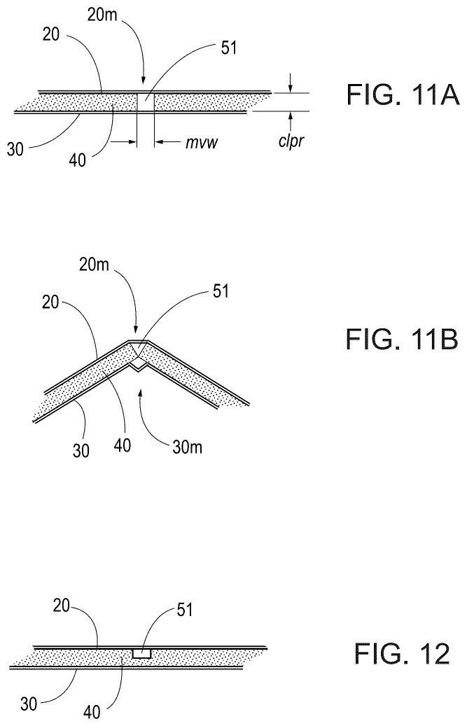

Referring to FIGS. 5, 11A and 11B, it may be desired that macro-voids along a path have a width mvw of 20 percent to 300 percent, preferably 50 percent to 150 percent, and even more preferably 75 percent to 125 percent, of the caliper clpr of the absorbent layer 40. Limiting macro-void width to a value within these ranges may help ensure that a desired level of flexibility to facilitate easy folding or creasing along the path of macro-voids is provided, while avoiding a macro-void of such a size that it is a gaping opening through the absorbent layer that permits menstrual fluid to move in an uncontrolled manner through it, to a space between the absorbent layer 40 and the backsheet 30. When the topsheet 20 is bonded to the absorbent layer 40 as described above, the portion 20m thereof bridging the macro-void constrains separation of the absorbent layer 40 across a macro-void 51 (as may be appreciated by a comparison of FIGS. 11A and 11B), upon bending about the path of macro-voids. This causes portions of the absorbent layer 40 on the inside of the crease or fold to be drawn together (as shown in FIG. 11B), desirably closing the opening or gap in the absorbent layer imparted by the macro-void 51. The portion 30m of backsheet 30 bridging the macro-void 51 may buckle in a manner such as, for example, that shown in FIG. 11B, to accommodate the folding or creasing. In the event of bending/creasing/folding in the direction opposite that shown in FIG. 11B, the roles of the topsheet and backsheet are reversed, and the gap in the absorbent layer at the macro-void is closed at the top, rather than at the bottom as shown in FIG. 11B. (For purposes herein, the width of a macro-void is its largest dimension at any location where the path traverses it, and along a direction normal to the path, and in the plane of the wearer-facing surface of the absorbent layer; see, e.g., dimension mvw shown in FIGS. 5 and 7. Absorbent layer caliper clpr is measured using the caliper measurement method provided below.)

In other examples but for similar purposes, macro-voids 51 may be formed in or imparted to an absorbent layer such that they do not extend in a z-direction through the entire z-direction depth/caliper of the absorbent layer 40, but rather, extend only partway through it; see the example shown in FIG. 12.

Macro-voids 51 may have a length of from about 2 mm to about 10 mm, or from about 2.5 mm to about 8 mm, or from about 3 mm to about 6 mm. In some configurations it may be preferable to have macro-voids 51 having a length of from about 2 mm to about 6 mm. Without being limited by theory, it is believed that macro-voids having a length of less than about 6 mm may help to minimize tearing during high speed processing.

A particularly suitable configuration may comprise macro-voids 51 of a forward path 50f having a length of about 4 mm with a gap length (G) between macro-voids of about 3 mm.

A particularly suitable configurations may comprise macro-voids 51 of a rearward path 50r having a length of about 3 mm with a gap length (G) of about 3 mm between macro-voids and/or between macro-voids and perforations 42 r that intersect the path of macro-voids.

In some configurations, a path of macro-voids 51 may have a ratio of a macro-void length to a gap length of from about 30:1 to about 1:5, or from about 10:1 to about 1:1.

Macro-voids 51 (and accompanying perforations 42f, 42r, if included) may be formed in or imparted to an absorbent layer 40 via any suitable method or process. In some configurations, they may be mechanically knife- or die-cut or punched through the absorbent layer or its precursor web or sheet. In some configurations, the process may include passing the layer (or precursor material in web or sheet) through a nip between a pair of die-cutting rollers configured to cut the desired arrangement of macro-voids (and perforations, if included) through the layer, web or sheet. In some examples, the same die-cutting rolls may be configured to cut out both perforations 42f, 42r, and macro-voids 51. In such examples, the resulting perimeter edges and interior walls of the macro-voids (and perforations, if included) will bear visible evidence of such mechanical cutting. In other examples, material may be removed from the absorbent layer (or precursor material in web or sheet form) via other methods configured to remove material along defined profiles forming the desired macro-void shapes, including but not limited to laser-cutting, fluid or water jet cutting or etching, etc. In other configurations, the material to form the absorbent layer may be formed in or on molds configured to mold the desired arrangement of macro-voids 51, with or without accompanying perforations 42f and/or 42r, into the absorbent layer (or precursor material). Mechanical cutting or punching, or fluid jet cutting or etching, may be preferred, however, because these methods leave dissected, open, exposed cells or pores in the absorbent layer material along the perimeter edges/walls of the macro-voids (and perforations, if included) exposed, which can enhance fluid distribution and absorbency.

Referring to FIG. 4I, in some configurations, it may be desired to add a shaped stiffener 60 beneath (i.e., to the outward-facing side of) absorbent layer 40. Stiffener 60 may be a partial layer component shaped and adapted to impart increased bending resistance to one or more selected regions of the pad occupied by absorbent layer 40, to cooperate with paths of macro-voids 51 to direct and/or more effectively induce hinging and folding or creasing there along, and to reduce or avoid folding/creasing of the pad in unwanted locations. Thus, in configurations that include a stiffener 60, it may be desired that stiffener 60 underlie one or more regions of absorbent layer 40 that are not occupied by macro-voids 51. In the example shown in FIG. 4I, a stiffener 60 underlies the region between the left and right paths of macro-voids 51, predominantly in the forward region of the pad (i.e., predominantly forward of the lateral axis 200) and straddling the longitudinal axis 100 and substantially symmetric thereacross. This feature may cause the absorbent layer 40 to more readily crease as intended along the left and right paths of macro-voids 51 shown, but also to resist excessive bending or creasing in unwanted locations, e.g., the middle region between the left and right paths. This may be desirable in configurations that include a pattern of apertures 42f (e.g., as shown in FIGS. 4G and 4H), between left and right paths of macro-voids 51, that may otherwise reduce stiffness of the absorbent layer 40 in that region. A stiffener 60 may be added to underlie a portion of or all of a pattern of apertures 42f.

A stiffener 60 may be formed of or Include an added, shaped section of nonwoven web material, film, tissue, paper, or any other added material that will impart added bending stiffness to selected and defined regions of the pad occupied by the absorbent layer 40. In some configurations, a stiffener 60 may simply be formed of or imparted by added or extra-basis weight deposit(s) of adhesive between the absorbent layer 40 and the backsheet 30, having a defined shape configured to impart stiffness to a correspondingly defined region of the pad. In other configurations, a stiffener 60 may be formed of or include an added, or increased basis-weight, application of fastening adhesive 35 applied to the outward-facing surface of the backsheet in regions where added stiffening is desired, as described above. In such configurations, the desired added stiffening will be effected when the user places and adheres the pad to her underpants for use/wear, through increased and/or relatively more continuous contact surface area and joining/adhesion of the pad structure with the fabric of the underpants, along that contact surface area and in the regions of desired added stiffness.

In other configurations, one or more portions or regions of the pad occupied by absorbent layer 40 may be subjected to selective mechanical deformation via deforming rollers as described in U.S. App. Ser. No. 63/424,979, in a manner that increases pliability/flexibility of the absorbent layer 40, and thus of the pad, in selected zones. Referring to FIG. 4D, in some configurations, a transition zone 40t may be subjected to such selective deformation, to increase its ability to flex and transition from the creasing/cupping configuration reflected in FIG. 4E to the creasing/inverted “V” configuration reflected in FIG. 4F. In other configurations, one or more peripheral edge regions of the absorbent layer 40 may be selectively deformed, to increase flexibility and pliability of the absorbent layer 40 about the regions proximate or adjacent its x-y plane perimeter edge 41. In some configurations, a portion or preferably a majority of the x-y planar surface area of the pad including the surface area of absorbent layer 40 disposed rearward of lateral axis 200 may be mechanically deformed as described above; this may enhance the ability of the pad to conform to and move with the user/wearer's body along the contours of the gluteal crevice. Additionally or alternatively, in some configurations, a portion or preferably a majority of the x-y planar surface area of the pad including the surface area of absorbent layer 40 disposed laterally outboard of left and right forward path legs 50f may be mechanically deformed as described above; this may enhance the ability of the pad to conform to and move with the user/wearer's body in creviced anterior portions of the crotch region, i.e., where the legs meet the lower torso. In such configurations, however, it may be desirable to avoid mechanical deformation of portions of the absorbent layer (40) that include paths of macro-voids 50f, 50r, or macro-voids 51 themselves, to avoid undesirable damage to the pad structure. When the pad is mechanically deformed as described in U.S. App. Ser. No. 63/424,979, the foam layer will bear visible evidence of such deformation in the form of tears or fractures along the z-direction, in the areas subjected to such deformation.

### Backsheet

The backsheet 30 may be positioned adjacent an outward-facing surface of the absorbent layer 40 and may be joined thereto by any suitable attachment methods. For example, the backsheet 30 may be secured to the absorbent layer 40 by a uniform continuous layer of adhesive, a patterned layer of adhesive, or an array of separate lines, spirals, or spots of adhesive. Alternatively, the attachment method may include heat bonds, pressure bonds, ultrasonic bonds, dynamic mechanical bonds, or any other suitable attachment mechanisms or combinations thereof. In other configurations, it is contemplated that the absorbent layer 40 is not joined directly to the backsheet 30.

The backsheet 30 may be impervious, or substantially impervious, to liquids (e.g., urine, menstrual fluid) and may be manufactured from a thin plastic film, although other flexible liquid impervious materials may also be used. As used herein, the term “flexible” refers to materials which are compliant and will readily conform to the general shape and contours of the human body. The backsheet 30 may prevent, or at least substantially inhibit, fluids absorbed and contained within the absorbent layer 40 from escaping and reaching articles of the wearer's clothing which may contact the pad 10 such as underpants and outer clothing. However, in some instances, the backsheet 30 may be made and/or adapted to permit vapor to escape from the absorbent layer 40 (i.e., the backsheet is made to be breathable), while in other instances the backsheet 30 may be made so as not to permit vapors to escape (i.e., it is made to be non-breathable). Thus, the backsheet 30 may comprise a polymeric film such as thermoplastic films of polyethylene or polypropylene. A suitable material for the backsheet 30 is a thermoplastic film having a thickness of from about 0.012 mm (0.5 mil) to about 0.051 mm (2.0 mils), for example. Any suitable backsheet known in the art may be utilized with the present invention.

Some suitable examples of backsheets are described in U.S. Pat. Nos. 5,885,265; 4,342,314; and 4,463,045. Suitable single layer breathable backsheets for use herein include those described for example in GB A 2184 389; GB A 2184 390; GB A 2184 391; U.S. Pat. Nos. 4,591,523, 3,989,867, 3,156,242; WO 97/24097; U.S. Pat. Nos. 6,623,464; 6,664,439 and 6,436,508.

The backsheet may have two layers: a first layer comprising a vapor permeable aperture-formed film layer and a second layer comprising a breathable microporous film layer, as described in U.S. Pat. No. 6,462,251. Other suitable examples of dual or multi-layer breathable backsheets for use herein include those described in U.S. Pat. Nos. 3,881,489, 4,341,216, 4,713,068, 4,818,600; EP 203 821, EP 710 471; EP 710 472, and EP 0 793 952.

### Caliper Measurement

The caliper, or thickness, of a test sample of an absorbent layer 40 is measured as the distance between a reference platform on which the sample rests and a pressure foot that exerts a specified amount of pressure onto the sample over a specified amount of time. All measurements are performed in a laboratory maintained at 23° C.±2 C.° and 50%±2% relative humidity and test samples are conditioned in this environment for at least 2 hours prior to testing.

Caliper is measured with a manually-operated micrometer equipped with a pressure foot capable of exerting a steady pressure of 2.0 kPa±0.01 kPa onto the test sample. The manually-operated micrometer is a dead-weight type instrument with readings accurate to 0.001 mm. A suitable instrument is Mitutoyo Series 543 ID-C Digimatic, available from VWR International, or equivalent. The pressure foot is a flat ground circular movable face with a diameter that is smaller than the test sample and capable of exerting the required pressure. A suitable pressure foot has a diameter of 25.4 mm, however a smaller or larger foot can be used depending on the size of the sample being measured. The test sample is supported by a horizontal flat reference platform that is larger than and parallel to the surface of the pressure foot. The system is calibrated and operated per the manufacturer's instructions.

Obtain a test sample, if necessary by removing it from an absorbent article. When excising the test sample from an absorbent article, use care to not impart any contamination or dimensional deformation to the test sample. The test sample is obtained from an area free of folds or wrinkles, and it must be larger than the pressure foot.

To measure caliper, first zero the micrometer against the horizontal flat reference platform. Place the test sample on the platform with the test location centered below the pressure foot. Gently lower the pressure foot with a descent rate of 3.0 mm+1.0 mm per second until the full pressure is exerted onto the test sample. Wait 5 seconds and then record the caliper of the test sample to the nearest 0.01 mm. In like fashion, repeat for a total of five replicate test samples. Calculate the arithmetic mean for all caliper measurements and report as Web Caliper to the nearest 0.01 mm.

### Examples

In view of the foregoing disclosure, the following examples are contemplated:
1. A feminine hygiene pad having a liquid-permeable topsheet (20), a liquid-impermeable backsheet (30), an absorbent layer (40) enveloped between the topsheet and the backsheet, a forward end, a rearward end, and a longitudinal axis (100) and a lateral axis (200), the axes having an intersection,
wherein the absorbent layer (40) comprises a layer of open-celled foam having a z-direction caliper and an outer perimeter (41) along an x-y plane, the foam layer having an arrangement of macro-voids (51) therein, the macro-voids having z-direction depth, wherein the arrangement of macro-voids defines paths (50f, 50r) that lie along an x-y planar surface of the foam layer and comprise:

a left forward path leg (50f) disposed predominantly forward of the lateral axis and beginning at a left central location proximate the intersection of the axes, and ending at a left outboard location disposed forward of the first left central location, and further outboard of the longitudinal axis than the first left central location; and a right forward path leg (50f) disposed predominantly forward of the lateral axis and beginning at a right central location proximate the intersection of the axes, and ending at a right outboard location disposed forward of the first right central location, and further outboard of the longitudinal axis than the first right central location.
2. The feminine hygiene pad of example 1 wherein the arrangement of macro-voids further comprises a rearward path leg (50r) disposed substantially along the longitudinal axis and predominantly rearward of the lateral axis.
3. The feminine hygiene pad of example 2 wherein one or more, preferably a majority, and more preferably all, of the macro-voids traversed by the rearward path leg (50r) do not intersect or coincide with any rearward elongate perforations (42r) present, which have their longest dimensions oriented predominantly along a lateral direction.
4. The feminine hygiene pad of any of the preceding examples wherein the absorbent layer has an arrangement of one or more rearward elongate perforations (42r) lying along the x-y planar surface of the pad, disposed predominantly rearward of the lateral axis, wherein the longest dimensions thereof are oriented predominantly along a lateral direction.
5. The feminine hygiene pad of any of the preceding examples wherein the absorbent layer has an arrangement of one or more forward perforations (42f) lying along the x-y planar surface of the pad, disposed predominantly between the left forward path leg and the right forward path leg.
6. The feminine hygiene pad of any of the preceding examples wherein the z-direction depth of at least some, preferably a majority, of the macro-voids extends entirely through the caliper of the foam layer.
7. The feminine hygiene pad of any examples 1-4 wherein the z-direction depth of at least some, preferably a majority, of the macro-voids extends only partially through the caliper of the foam layer, and preferably, wherein the z-direction depth is 40 percent to 70 percent of the caliper of the foam layer, and preferably, wherein the z-direction depth begins and extends from a wearer-facing side of the foam layer.
8. The feminine hygiene pad of any of the preceding examples wherein one or more of the path legs traverses at least four, preferably at least five macro-voids.
9. The feminine hygiene pad of any of the preceding examples wherein each of the macro-voids along each of the path legs has a width, and the width is equal to 20 percent to 200 percent, more preferably 50 percent to 150 percent, and even more preferably 75 percent to 125 percent, of the z-direction caliper.
10. The feminine hygiene pad of any of the preceding examples wherein the open-celled foam is one or more of a HIPE foam and a polyurethane foam, preferably a HIPE foam.
11. The feminine hygiene pad of any of the preceding examples wherein the perimeter tapers from a laterally wider x-y dimension in a rearward portion of the pad, to a laterally narrower x-y dimension in a forward portion of the pad.
12. The feminine hygiene pad of any of the preceding examples comprising a stiffener (60) underlying one or more portions of the absorbent layer.
13. The feminine hygiene pad of example 12 wherein the stiffener does not underlie a majority of the macro-voids (51).
14. The feminine hygiene pad of either of examples 12 or 13 wherein the stiffener has an x-y surface area disposed predominantly forward of the lateral axis (200).
15. The feminine hygiene pad of any of examples 12-14 wherein the stiffener has an x-y surface area disposed at least partially, and more preferably, predominantly, between the left and right forward path legs (50f).
16. The feminine hygiene pad of any of the preceding examples wherein the foam layer has an x-y planar surface area, and a portion, and preferably a majority, of the pad including the foam layer x-y planar surface area disposed rearward of the lateral axis (200) is mechanically deformed.
17. The feminine hygiene pad of any of the preceding examples wherein the foam layer has an x-y planar surface area, and a portion, and preferably a majority, of the pad including the foam layer x-y planar surface area disposed laterally outboard of the left and right forward path legs (50f) is mechanically deformed.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety to the extent not inconsistent herewith and unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A feminine hygiene pad comprising:
a forward end, a rearward end, and a longitudinal axis and a lateral axis, the axes having an intersection;
a liquid-permeable topsheet;
a liquid-impermeable backsheet; and
an absorbent layer disposed between the topsheet and the backsheet;
wherein the absorbent layer comprises a layer of open-celled foam having a z-direction caliper and an outer perimeter along an x-y plane, the layer of open-celled foam comprises an arrangement of macro-voids therein, the macro-voids having a z-direction depth, wherein the arrangement of macro-voids defines paths that lie along an x-y planar surface of the layer of open-celled foam and comprise:
a left forward path leg disposed predominantly forward of the lateral axis and beginning at a left central location proximate the intersection of the axes, and ending at a left outboard location disposed forward of the first left central location, and further outboard of the longitudinal axis than the first left central location; and
a right forward path leg disposed predominantly forward of the lateral axis and beginning at a right central location proximate the intersection of the axes, and ending at a right outboard location disposed forward of the first right central location, and further outboard of the longitudinal axis than the first right central location.

2. The feminine hygiene pad of claim 1, wherein the arrangement of macro-voids further comprises a rearward path leg disposed substantially along the longitudinal axis and predominantly rearward of the lateral axis.

3. The feminine hygiene pad of claim 1, wherein the absorbent layer comprises an arrangement of one or more forward perforations lying along the x-y planar surface of the pad, wherein the forward perforations are disposed between the left forward path leg and the right forward path leg.

4. The feminine hygiene pad of claim 1, wherein the z-direction depth of a portion of the macro-voids extends entirely through the caliper of the foam layer.

5. The feminine hygiene pad of claim 1, wherein the z-direction depth of a portion of the macro-voids is about 40 percent to about 70 percent of the caliper of the foam layer, wherein the z-direction depth begins and extends from a wearer-facing side of the foam layer.

6. The feminine hygiene pad of claim 1, wherein one or more of the path legs traverses at least four macro-voids.

7. The feminine hygiene pad of claim 1, wherein each of the macro-voids along each of the path legs has a width, and the width is equal to about 20 percent to about 200 percent of the z-direction caliper.

8. The feminine hygiene pad of claim 1, wherein the open-celled foam is one or more of a HIPE foam and a polyurethane foam.

9. The feminine hygiene pad of claim 1, wherein the arrangement of macro-voids further comprises a second left forward path leg disposed outboard of the left forward path leg.

10. The feminine hygiene pad of claim 9, wherein the second left forward path leg extends substantially parallel to the left forward path leg.

11. The feminine hygiene pad of claim 10, wherein the left forward path leg and the second left forward path leg are separated by a distance of from about 2 mm to about 4 mm.

12. The feminine hygiene pad of claim 9, wherein the arrangement of macro-voids further comprises a second right forward path leg.

13. The feminine hygiene pad of claim 12, wherein the second right forward path leg extends substantially parallel to the right forward path leg.

14. The feminine hygiene pad of claim 1, further comprising a stiffener underlying one or more portions of the absorbent layer.

15. A feminine hygiene pad comprising:
a forward end, a rearward end, and a longitudinal axis and a lateral axis, the axes having an intersection;
a liquid-permeable topsheet;
a liquid-impermeable backsheet; and
an absorbent layer disposed between the topsheet and the backsheet;
wherein the absorbent layer comprises a layer of open-celled foam having a z-direction caliper and an outer perimeter along an x-y plane, the layer of open-celled foam comprises an arrangement of macro-voids therein, the macro-voids having a z-direction depth, wherein the arrangement of macro-voids defines paths that lie along an x-y planar surface of the layer of open-celled foam and comprise:
a left forward path leg disposed predominantly forward of the lateral axis and beginning at a left central location proximate the intersection of the axes, and ending at a left outboard location disposed forward of the first left central location, and further outboard of the longitudinal axis than the first left central location; and
a right forward path leg disposed predominantly forward of the lateral axis and beginning at a right central location proximate the intersection of the axes, and ending at a right outboard location disposed forward of the first right central location, and further outboard of the longitudinal axis than the first right central location;
wherein at least one of the left forward path leg and the right forward path leg has a ratio of a macro-void length to a gap length of from about 30:1 to about 1:5.

16. The feminine hygiene pad of claim 15, wherein the arrangement of macro-voids further comprises a rearward path leg disposed substantially along the longitudinal axis and predominantly rearward of the lateral axis.

17. The feminine hygiene pad of claim 16, wherein the arrangement of macro-voids further comprises a second left forward path leg disposed outboard of the left forward path leg and a second right forward path leg disposed outboard of the right forward path leg.

18. The feminine hygiene pad of claim 17, wherein the left forward path leg and the second left forward path leg are separated by a distance of from about 2 mm to about 4 mm.

19. The feminine hygiene pad of claim 15, wherein the open-celled foam is one or more of a HIPE foam and a polyurethane foam.

20. The feminine hygiene pad of claim 15, further comprising a stiffener underlying one or more portions of the absorbent layer, wherein the stiffener has an x-y surface area disposed predominantly forward of the lateral axis.
